(19) United States
(12) Patent Application Publication
(10) Pub. No.: US 2022/0143150 A1
(43) Pub. Date: May 12, 2022

(54) GLUCAGON-LIKE PEPTIDE-1 (GLP-1) AGONIST ANALOG, PROCESS OF PREPARATION AND USES THEREOF

(71) Applicant: ENZENE BIOSCIENCES LIMITED, Pune (IN)

(72) Inventors: Himanshu GADGIL, Pune (IN); Abir BANERJEE, Pune (IN); Harshita LONDHE, Pune (IN); Deepali MAGDUM, Kolhapur (IN); Daniel LEVIN, La Canada, CA (US); Sandeep SINGH, Mumbai (IN)

(73) Assignee: ENZENE BIOSCIENCES LIMITED, Pune (IN)

(21) Appl. No.: 17/428,872

(22) PCT Filed: Feb. 6, 2020
(86) PCT No.: PCT/IB2020/050957

§ 371 Date: Aug. 5, 2021

(30) Foreign Application Priority Data
Feb. 6, 2019 (IN) 201921004787

(51) Int. Cl. A61K 38/26; A61P 3/10

(52) U.S. Cl. CPC A61K 38/26; A61P 3/10

(57) ABSTRACT

The present disclosure relates to an analog of glucagon-like peptide-1 (glp-1) receptor agonist. The present disclosure provides analogs of glucagon-like peptide-1 (glp-1) receptor agonist wherein, the amino acid at position 2 of the glucagon-like peptide-1 (glp-1) receptor agonist is replaced with D-Alanine. The analogs of glucagon-like peptide-1 (glp-1) have one or more properties of prolonged half-life, better pharmacokinetic profile, retained biological activity, and being advantageous for relieving the patient's burden by reducing the dosing frequency and dose. The present disclosure further provides processes for preparing synthetic glucagon-like peptide-1 (glp-1) analogs.

### FIELD OF THE INVENTION

The present disclosure relates to an analog of glucagon-like peptide-1 (glp-1). More particularly, the present disclosure relates to an analog of glucagon-like peptide-1 (glp-1) receptor agonist wherein, the amino acid at position 2 of the native glucagon-like peptide-1 (glp-1) receptor agonist is replaced with D-Alanine. The present invention further relates to analogs of glucagon-like peptide-1 (glp-1) having one or more properties of prolonged half-life, better pharmacokinetic profile, retained biological activity, and being advantageous for relieving the patient's burden by reducing the dosing frequency and dose. Specifically, the present invention relates to synthetic glucagon-like peptide-1 (glp-1) analogs obtained from different peptide synthesis processes and processes for preparing synthetic glucagon-like peptide-1 (glp-1) analogs.

### BACKGROUND OF THE INVENTION

Background description includes information that may be useful in understanding the present invention. It is not an admission that any of the information provided herein is prior art or relevant to the presently claimed invention, or that any publication specifically or implicitly referenced is prior art.

As a drug class, long-acting GLP-1 receptor agonists increase glycemic control in patients with type 2 diabetes with a low risk of hypoglycemia because of their glucose-dependent mechanism of action. Glucagon-like peptide-1 (GLP-1) is produced by the gut, and in a glucose-dependent manner stimulates insulin secretion while inhibiting glucagon secretion, reduces appetite and energy intake, and delays gastric emptying. This drug class has also been demonstrated to promote weight loss and reduce SBP, which could be of benefit to patients with type 2 diabetes, reducing their cardiovascular risk. Furthermore, although nausea is a common side effect with long acting GLP-1 receptor agonists, it tends to be transient and, overall, long-acting GLP-1 receptor agonists are generally well tolerated. Thus, long-acting GLP-1 receptor agonists may provide an effective therapeutic option for individuals with type 2 diabetes and are well placed to meet the standard of care guidelines set by the ADA in treating more than just blood glucose.

GLP-1 is susceptible to cleavage at position 2 (alanine) by the ubiquitous dipeptidyl peptidase (DPP)-4, which occurs almost immediately upon secretion of GLP-1, rendering it a short half-life of <2 minutes (Gupta V., Indian J EndocrMetab 2013, 17, 413-21).

Many GLP-1 agonists were developed by modifications to natural GLP-1 to overcome the problem of its short half-life. One of the approaches used was substitution of one or more amino acids of the GLP-1 polypeptide and attachment of a lipophilic substituent to these peptides. These lipophilic substituted GLP-1 agonists showed protracted action when injected. U.S. Pat. No. 6,268,343 disclosed such fatty acid acylated GLP-1 agonists.

One particular example of GLP-1 analog is Liraglutide. Liraglutide is an acylated glucagon-like peptide-1 (GLP-1) agonist, derived from human GLP-1-(7-37), a less common form of endogenous GLP-1. Liraglutide has a short plasma half-life (9-15 hours) and novel methods have been developed to augment its half-life, such that its anti-hyperglycemic effects can be exploited. A once-daily injection to the diabetic patient is required for the treatment.

Semaglutideis also a GLP-1 analog recently been registered to treat type 2 diabetes. Semaglutide has two amino acid substitutions compared to human GLP-1 (Aib(8), Arg(34)) and is derivatized at lysine 26.

Several studies have been conducted evaluating semaglutide's pharmacokinetics as a once-weekly subcutaneous injection. As a dose of 0.5 or 1 mg, semaglutide has a half-life of 7 days; therefore, it would reach steady state in 4-5 weeks. However, there are few drug interactions and dose adjustments are necessary. Besides, similar to other GLP-1 RAs, semaglutide can delay gastric emptying and may impact the absorption of oral medications. Although, semaglutide may be a useful drug in subjects with Type 2 Diabetes, however, it has been observed to increase retinopathy to a small extent. Also, it is not known whether semaglutide will improve cardiovascular outcomes in other populations including those with lower ages, HbA1c values, and body weights similar to those included in the unsuccessful clinical outcome trials with the GLP-1R agonists, lixisenatide and exenatide. Other challenge associated with the semaglutide is the dosage of semaglutide required for the oral formulation, which is much higher for oral semaglutide than it is for branded Ozempic injectable semaglutide. Oral semaglutide required 14 mg of semaglutide per dose to achieve the effects described in the trial, whereas Ozempic required only 0.5 mg to achieve slightly better results. The discrepancy is the result of most of the active oral drug being digested by the stomach and small intestine and only a small fraction getting through the intestinal wall on the way to the liver in order to achieve therapeutic results

So far, research suggests that semaglutide may yield greater blood sugar control and more weight loss, nonetheless there are some drawbacks such as injecting the medication, or much higher dosage of semaglutide required for the oral formulation, common side effects, increased risk of retinopathy, and potential cost.

However, analogs of GLP-1 with enhanced half-life, leading to increased bioavailability while retaining its clinical efficacy have not been explored fully.

There is, therefore, a need to develop GLP-1 analogs that can overcome deficiencies associated with the known arts.

Therefore, still there is a need to provide the analog of GLP-1, which can overcome one or more of the above disadvantages, so that the promising candidate like GLP-1 analog such as liraglutide and semaglutide can get the place they deserve in diabetes and other treatment(s).

### OBJECTS OF THE INVENTION

An object of the present disclosure to provide an analog of glucagon-like peptide-1 (glp-1) receptor agonist, which may overcome one or more of the shortcomings of the existing analog of glucagon-like peptide-1 (glp-1).

It is an object of the present disclosure to provide an analog of glucagon-like peptide-1 (glp-1) receptor agonist, wherein, the amino acid at position 2 of the native glucagon-like peptide-1 (glp-1) receptor agonist is replaced with D-alanine.

It is an object of the present disclosure to provide a process for preparation of an analog of glucagon-like peptide-1 (glp-1) receptor agonist, wherein the amino acid at position 2 of the native glucagon-like peptide-1 (glp-1) receptor agonist is replaced with D-alanine.

It is an object of the present disclosure to provide analogs of glucagon-like peptide-1 (glp-1) which can retain the biological activity of the peptides, prolong the half-life, have a better pharmacokinetic profile, and be advantageous for relieving the patient's burden by reducing the dosing frequency and dose.

It is an object of the present disclosure to provide analogs of liraglutide and semaglutide that can overcome one or more deficiencies found in the existing art.

It is an object of the present disclosure to provide analogs of liraglutide and semaglutide with one or more properties of having prolonged half-life, better pharmacokinetic profile and still able to retain respective specific biological activity, and being advantageous for relieving the patient's burden by reducing the dosing frequency and dose.

Another object of the present invention is to provide a synthetic analog of liraglutide and semaglutide that can be easily synthesized.

### SUMMARY OF THE INVENTION

In an aspect the present disclosure provides an analog of glucagon-like peptide-1 (glp-1) receptor agonist, which may overcome one or more of the shortcomings of the existing glucagon-like peptide-1 (glp-1).

In one aspect the present disclosure provides an analog of glucagon-like peptide-1 (glp-1) receptor agonist, wherein, the amino acid at position 2 of the native glucagon-like peptide-1 (glp-1) receptor agonist is replaced with D-Alanine.

In one aspect the present disclosure provides an analog of glucagon-like peptide-1 (glp-1) receptor agonist, wherein, the glucagon-like peptide-1 (glp-1) receptor agonist is liraglutide or semaglutide.

In one aspect the present disclosure provides a process for preparation of an analog of glucagon-like peptide-1 (glp-1) receptor agonist, wherein, the amino acid at position 2 of the native glucagon-like peptide-1 (glp-1) receptor agonist is replaced with D-Alanine.

In another aspect, the present disclosure provides an analog of liraglutide wherein L-Alanine amino acid at position 2 of the native glucagon-like peptide-1 (glp-1) receptor agonist is replaced with D-Alanine.

In another aspect, the present disclosure provides an analog of semaglutide, wherein the Aib (Amino isobutyric acid) amino acid at position 2 of the native glucagon-like peptide-1 (glp-1) receptor agonist is replaced with D-Alanine.

In another aspect, the present disclosure provides a method of reducing glucose levels in a patient in need thereof, comprising administering analog of liraglutide or semaglutide analog of the present disclosure.

In another aspect, the present disclosure provides a long acting liraglutide analog for once weekly or biweekly or monthly administration.

In one more aspect, the present invention relates to the process for the preparation of D-Liraglutide with amino acid at position 2 of the native liraglutide replaced with D-Alanine, in which the process comprises the steps of:
a) anchoring Fmoc-Gly-OH to a resin and capping it;
b) selectively deprotecting the amino group;
c) sequential coupling of the fragments Fmoc-Arg(Pbf)OH, Fmoc-Gly-OH, Fmoc-Arg(Pbf)OH, Fmoc-Val-OH, Fmoc-Leu-OH, Fmoc-Trp(Boc)-OH, Fmoc-Ala-OH, Fmoc-Ile-OH, Fmoc-Phe-OH, Fmoc-Glu(OtBu)-OH, Fmoc-Lys(Dde)-OH, Fmoc-Ala-OH, Fmoc-Ala-OH, Fmoc-Gln(Trt)-OH, Fmoc-Glu(OtBu)-OH, Fmoc-Gly-OH, Fmoc-Leu-OH, Fmoc-Tyr(tBu)-OH, Fmoc-Ser(tBu)-OH, Fmoc-Ser(tBu)-OH, Fmoc-Val-OH, Fmoc-Asp(OtBu)-OH, Fmoc-Ser(tBu)-OH, Fmoc-Thr(tBu)-OH, Fmoc-Phe-OH, Fmoc-Thr(tBu)-OH, Fmoc-Phe-OH, Fmoc-Thr(tBu)-OH, Glu(OtBu)-OH, Fmoc-Gly-OH, Fmoc-D-Ala-OH and Boc-His(Trt)-OH;
d) removing of the lysine side chain protecting group Dde, followed by coupling with Fmoc-Glu-OtBu, followed by Fmoc deprotection and coupling with palmitic acid; and
e) cleaving the peptide from the resin to obtain linear D-Liraglutide;

In one aspect, the process optionally comprises purifying the D-Liraglutide to provide purified D-Liraglutide.

In one aspect, the present disclosure provides a process for preparation of D-Semaglutide analogue with amino acid at position 2 of the native semaglutide replaced with D-Alanine, in which the process comprises the steps of:
a) anchoring Fmoc-Gly-OH to a resin and capping it;
b) selectively deprotecting the amino group;
c) sequential coupling of the fragments Fmoc-Arg(Pbf)OH, Fmoc-Gly-OH, Fmoc-Arg(Pbf)OH, Fmoc-Val-OH, Fmoc-Leu-OH, Fmoc-Trp(Boc)-OH, Fmoc-Ala-OH, Fmoc-Ile-OH, Fmoc-Phe-OH, Fmoc-Glu(OtBu)-OH, Fmoc-Lys(Dde)-OH, Fmoc-Ala-OH, Fmoc-Ala-OH, Fmoc-Gln(Trt)-OH, Fmoc-Glu(OtBu)-OH, Fmoc-Gly-OH, Fmoc-Leu-OH, Fmoc-Tyr(tBu)-OH, Fmoc-Ser(tBu)-OH, Fmoc-Ser(tBu)-OH, Fmoc-Val-OH, Fmoc-Asp(OtBu)-OH, Fmoc-Ser(tBu)-OH, Fmoc-Thr(tBu)-OH, Fmoc-Phe-OH, Fmoc-Thr(tBu)-OH, Fmoc-Phe-OH, Fmoc-Thr(tBu)-OH, Glu(OtBu)-OH, Fmoc-Gly-OH, Fmoc-D-Ala-OH and Boc-His(Trt)-OH;
d) removing the lysine side chain protecting group Dde, followed by coupling with Fmoc-PEG2-CH₂—COOH sequences, Fmoc-Glu-OtBu followed by Fmoc deprotection and coupling with oxaoctadecanoic acid; and
e) cleaving the peptide from the resin to obtain linear D-Semaglutide.

In one aspect, the process optionally comprises purifying D-Semaglutide to provide purified D-Semaglutide.

In another aspect, the present disclosure provides a suitable dosage form comprising the GLP-1 analog of the present disclosure by. Such dosage form can be suitable for administration through oral or parenteral route.

In another aspect, the present disclosure provides a suitable dosage form comprising of the analog of Liraglutide, or Semaglutide provided in accordance with the present disclosure. Such dosage form to be suitable for administration through oral or parenteral route.

In one aspect the present disclosure provides a method of reducing glucose levels in a patient in need thereof, comprising administering the GLP-1 analog of the present disclosure in a therapeutically effective amount.

In one aspect the present disclosure provides a method of reducing glucose levels in a patient in need thereof, comprising administering the analog of Liraglutide, or Semaglutide of the present disclosure in a therapeutically effective amount.

Various objects, features, aspects and advantages of the inventive subject matter will become more apparent from the following detailed description of preferred embodiments.

### BRIEF DESCRIPTION OF DRAWINGS THE INVENTION

The following drawings form part of the present specification and are included to further illustrate aspects of the present disclosure. The disclosure may be better understood by reference to the drawings in combination with the detailed description of the specific embodiments presented herein.

FIG. 1 is a flow-chart depicting a protocol for the preparation of D-Liraglutide comprising the steps as shown in scheme 1 as per one of the exemplary embodiments of the present disclosure.

FIG. 2 is a flow-chart depicting a protocol for the preparation of D-Semaglutide comprising the steps as shown in scheme 2 as per one of the exemplary embodiments of the present disclosure.

FIG. 3 is RP-HPLC profile for Liraglutide

FIG. 4 is RP-HPLC profile for D-Liraglutideas per one of the exemplary embodiments of the present disclosure.

FIG. 5 is a chromatogram profile for purification of Liraglutide

FIG. 6 is a chromatogram profile for purification of D-Liraglutideas per one of the exemplary embodiments of the present disclosure.

FIG. 7 is RP-HPLC profile for purified Liraglutide

FIG. 8 is RP-HPLC profile for purified D-Liraglutide as per one of the exemplary embodiments of the present disclosure.

FIG. 9 is a graph showing comparative EC50 values of reference product Victoza, Liraglutide and D-Liraglutide, wherein SPL1 represents Liraglutide and SPL2 represents D-Liraglutideas per one of the exemplary embodiments of the present disclosure.

FIG. 10 is comparative PK profiles of Liraglutide and D-Liraglutide wherein CL represents Liraglutide and TL represents D-Liraglutideas per one of the exemplary embodiments of the present disclosure.

FIG. 11 are graphs, wherein FIG. 11(a) showing PK Profile of orally administered D-Liraglutide as per one of the exemplary embodiments of the present disclosure; and FIG. 11(b) showing PK Profile of subcutaneously administered reference product Victoza and D-Liraglutide as per one of the exemplary embodiments of the present disclosure.

### DETAILED DESCRIPTION

The following is a detailed description of embodiments of the disclosure. The embodiments are in such detail as to clearly communicate the disclosure. However, the amount of detail offered is not intended to limit the anticipated variations of embodiments; on the contrary, the intention is to cover all modifications, equivalents, and alternatives falling within the spirit and scope of the present disclosure as defined by the appended claims.

All publications herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

Reference throughout this specification to “one embodiment” or “an embodiment” means that a particular feature, structure or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

In some embodiments, the numbers expressing quantities of ingredients, properties such as concentration, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise.

Unless the context requires otherwise, throughout the specification which follow, the word “comprise” and variations thereof, such as, “comprises” and “comprising” are to be construed in an open, inclusive sense that is as “including, but not limited to.”

The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided with respect to certain embodiments herein is intended merely to better illustrate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

The description that follows, and the embodiments described therein, is provided by way of illustration of an example, or examples, of particular embodiments of the principles and aspects of the present disclosure. These examples are provided for the purposes of explanation, and not of limitation, of those principles and of the disclosure.

It should also be appreciated that the present disclosure can be implemented in numerous ways, including as a system, a method or a device. In this specification, these implementations, or any other form that the invention may take, may be referred to as processes. In general, the order of the steps of the disclosed processes may be altered within the scope of the invention.

The headings and abstract of the invention provided herein are for convenience only and do not interpret the scope or meaning of the embodiments.

The following discussion provides many example embodiments of the inventive subject matter. Although each embodiment represents a single combination of inventive elements, the inventive subject matter is considered to include all possible combinations of the disclosed elements. Thus, if one embodiment comprises elements A, B, and C, and a second embodiment comprises elements B and D, then the inventive subject matter is also considered to include other remaining combinations of A, B, C, or D, even if not explicitly disclosed.

Various terms as used herein are shown below. To the extent a term used in a claim is not defined below, it should be given the broadest definition persons in the pertinent art have given that term as reflected in printed publications and issued patents at the time of filing.

The term, “analog” as used herein refers to a compound having a structure similar to that of another compound, but differing from it in respect to a certain component. Such analogs can have very different physical, chemical, biochemical, or pharmacological properties.

Abbreviations as used herein refers to the following full forms:
Boc: t-Butyloxycarbonyl
DCM: Dichloromethane
Dde: 1-(4,4-dimethyl-2,6-dioxocyclohex-1-ylidene)ethyl
DIC: N, N′-Diisopropylcarbodiimide
DIPE A: Diisopropylethylamine
DMF: Dimethylformamide
DODT: 2,2′-(Ethylenedioxy)diethanethiol
Fmoc: 9-Fluorenylmethoxycarbonyl
HBTU: Hexafluorophosphate benzotriazole tetramethyluronium
HOBt: N-Hydroxybenzotriazole
HPLC: High Performance Liquid Chromatography
MTBE: Methyl-t-butyl ether
OtBu: tert-Butyl ester
tBu: tert-Butyl
TFA: Trifluoroacetic acid
Trt: Trityl
2-CTC: 2-Chlorotrityl chloride
HCl: Hydrochloric acid
mL: millilitre
g: gram
° C.: degree Celsius
h: hour
min: minutes
IPA: Isopropanol
vol: Volumes
RT: Room Temperature
Mmol: Milli mole
TIPS: Triisopropylsilane
A°: Angstrom
HPLC: High Performance Liquid Chromatography

The present disclosure relates to synthetic analogs of (glp-1) receptor agonist.

In general embodiment the present disclosure provides analogs of glucagon-like peptide-1 (glp-1) receptor agonist.

In certain embodiments the present disclosure provides analogs of glucagon-like peptide-1 (glp-1) receptor agonist, wherein the amino acid at position 2 of the native glucagon-like peptide-1 (glp-1) receptor agonist is replaced with D-Alanine.

The analog of glucagon-like peptide-1 (glp-1) receptor agonist, wherein the amino acid at position 2 of the native glucagon-like peptide-1 (glp-1) receptor agonist is replaced with D-Alanine is advantageous over the native glucagon-like peptide-1 (glp-1) receptor agonist for example it can have better bioavailability and enhanced efficacy than the respective glucagon-like peptide-1 (glp-1) receptor agonist.

In one embodiment the present disclosure provides an analog of glucagon-like peptide-1 (glp-1) receptor agonist, wherein the glucagon-like peptide-1 (glp-1) receptor agonist is a liraglutide or semaglutide.

In one embodiment, the present disclosure discloses synthetic analog of liraglutide, which can retain its biological activity.

In one embodiment, the present disclosure discloses synthetic analog of semaglutide, which can retain its biological activity.

The synthetic analog of liraglutide is also referred to herein as analog of GLP-1, GLP-A analog, analog of liraglutide, liraglutide analog, or D-liraglutide, analog of glucagon-like peptide-1 (glp-1) receptor agonist and such expressions are used interchangeably throughout.

The synthetic analog of semaglutide is also referred to herein as analog of GLP-1, GLP-A analog, analog of semaglutide, semaglutide analog, or D-semaglutide, analog of glucagon-like peptide-1 (glp-1) receptor agonist and such expressions are used interchangeably throughout.

In another embodiment, the present disclosure discloses synthetic analog of liraglutide that can be easily synthesized by solid phase peptide synthesis.

In another embodiment, the present disclosure discloses synthetic analog of semaglutide that can be easily synthesized by solid phase peptide synthesis.

In one embodiment the present disclosure provides a process for preparation of a synthetic analog of glucagon-like peptide-1 (glp-1) receptor agonist, wherein, the amino acid at position 2 of the native glucagon-like peptide-1 (glp-1) receptor agonist is replaced with D-Alanine.

In one embodiment the present disclosure provides a process for preparation of a synthetic analog of liraglutide wherein, the amino acid at position 2 of the native glucagon-like peptide-1 (glp-1) receptor agonist is replaced with D-Alanine.

In one embodiment the present disclosure provides a process for the preparation of D-Liraglutide with amino acid at position 2 of the native liraglutide replaced with D-Alanine, in which the process comprises the steps:
a) anchoring Fmoc-Gly-OH to a resin and capping it;
b) selectively deprotecting the amino group;
c) sequential coupling of the fragments Fmoc-Arg(Pbf)OH, Fmoc-Gly-OH, Fmoc-Arg(Pbf)OH, Fmoc-Val-OH, Fmoc-Leu-OH, Fmoc-Trp(Boc)-OH, Fmoc-Ala-OH, Fmoc-Ile-OH, Fmoc-Phe-OH, Fmoc-Glu(OtBu)-OH, Fmoc-Lys(Dde)-OH, Fmoc-Ala-OH, Fmoc-Ala-OH, Fmoc-Gln(Trt)-OH, Fmoc-Glu(OtBu)-OH, Fmoc-Gly-OH, Fmoc-Leu-OH, Fmoc-Tyr(tBu)-OH, Fmoc-Ser(tBu)-OH, Fmoc-Ser(tBu)-OH, Fmoc-Val-OH, Fmoc-Asp(OtBu)-OH, Fmoc-Ser(tBu)-OH, Fmoc-Thr(tBu)-OH, Fmoc-Phe-OH, Fmoc-Thr(tBu)-OH, Fmoc-Phe-OH, Fmoc-Thr(tBu)-OH, Glu(OtBu)-OH, Fmoc-Gly-OH, Fmoc-D-Ala-OH and Boc-His(Trt)-OH;
d) removing of the lysine side chain protecting group Dde, followed by coupling with Fmoc-Glu-OtBu, followed by Fmoc deprotection and coupling with palmitic acid; and
e) cleaving the peptide from the resin to obtain linear D-Liraglutide.

In one aspect, the process optionally comprises purifying D-Liraglutide to provide purified D-Liraglutide.

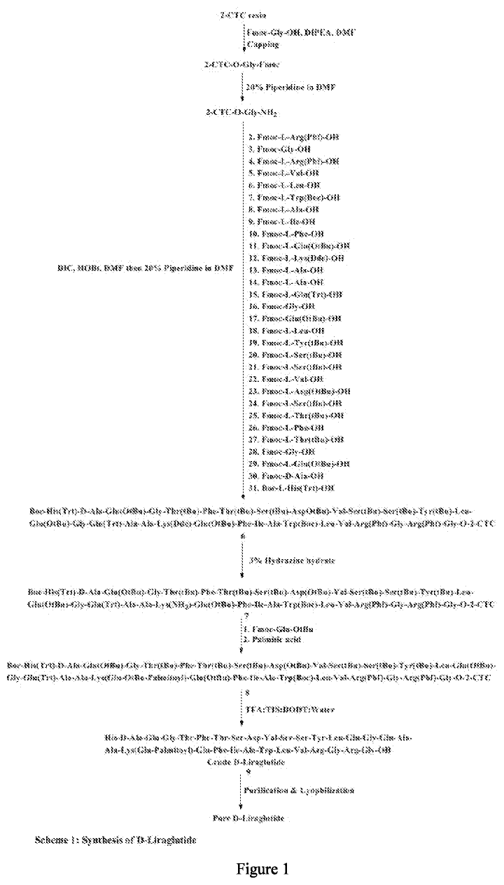

In another embodiment the present disclosure provides the process for the preparation of D-Liraglutide which comprises the steps as shown in scheme 1 (FIG. 1).

In one embodiment, the present disclosure provides a process for preparation of D-Semaglutide analogue with amino acid at position 2 of the native semaglutide replaced with D-Alanine, in which the process comprises steps of:
a) anchoring Fmoc-Gly-OH to a resin and capping it;
b) selectively deprotecting the amino group;
c) sequential coupling of the fragments Fmoc-Arg(Pbf)OH, Fmoc-Gly-OH, Fmoc-Arg(Pbf)OH, Fmoc-Val-OH, Fmoc-Leu-OH, Fmoc-Trp(Boc)-OH, Fmoc-Ala-OH, Fmoc-Ile-OH, Fmoc-Phe-OH, Fmoc-Glu(OtBu)-OH, Fmoc-Lys(Dde)-OH, Fmoc-Ala-OH, Fmoc-Ala-OH, Fmoc-Gln(Trt)-OH, Fmoc-Glu(OtBu)-OH, Fmoc-Gly-OH, Fmoc-Leu-OH, Fmoc-Tyr(tBu)-OH, Fmoc-Ser(tBu)-OH, Fmoc-Ser(tBu)-OH, Fmoc-Val-OH, Fmoc-Asp(OtBu)-OH, Fmoc-Ser(tBu)-OH, Fmoc-Thr(tBu)-OH, Fmoc-Phe-OH, Fmoc-Thr(tBu)-OH, Fmoc-Phe-OH, Fmoc-Thr(tBu)-OH, Glu(OtBu)-OH, Fmoc-Gly-OH, Fmoc-D-Ala-OH and Boc-His(Trt)-OH;
d) removing the lysine side chain protecting group Dde, followed by coupling with Fmoc-PEG2-CH₂—COOH sequences, Fmoc-Glu-OtBu followed by Fmoc deprotection and coupling with oxaoctadecanoic acid; and
e) cleaving the peptide from the resin to obtain linear D-Semaglutide.

In one embodiment the process optionally comprises purifying D-Semaglutide to provide purified D-Semaglutide.

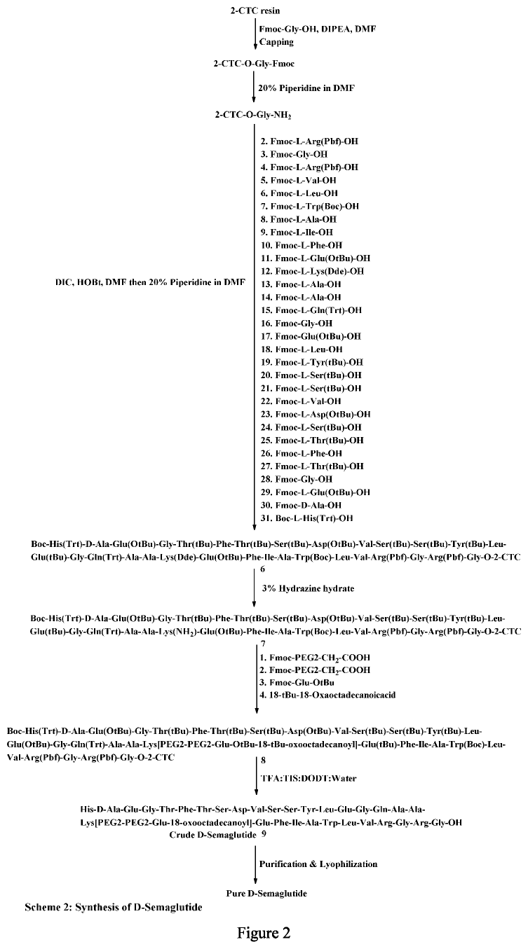

In one embodiment the present disclosure provides a process for the preparation of D-Semaglutide which comprises the steps as shown in scheme 2 (FIG. 2).

In one embodiment the solid phase is a resin.

In one embodiment the resin is selected from but not limited to 2-Chlorotrityl chloride (2-CTC), Sasrin, TentaGel S, TentaGel TGA, Rink, Wang, AmphiSpheres and other suitable resins.

In one embodiment coupling agent is selected from but not limited to 1-Hydroxybenzotriazole (HOBt), N,N-diisopropylcarbodiimide (DIC), Hexafluorophosphate Benzotriazole Tetramethyl Uronium (HBTU), N,N-Diisopropylethylamine (DIPEA), benzotriazol-1-yl-oxy-tris(dimethyl-amino)-phosphonium hexafluorophosphate (BOP), 0-(7-azabenzotriazol-1-yl)-1,1,3,3-tetramethyluronium hexafluorophosphate (HATU) and combination thereof.

In one embodiment the solvent for coupling reaction is selected from but not limited to DMF, pyridine, acetic anhydride, methanol, ethanol, isopropanol, dichloroethane, 1,4-dioxane, 2-methyl tetrahydrofuran, N-methyl-2-pyrrolidinone (NMP), ethyl acetate, acetonitrile, acetone, and the like or combination thereof.

In one embodiment the amino group can be selectively deprotected by methods known in the art for example by using a mixture of piperidine, DBU and dichloromethane in an appropriate solvent such as DMF.

In one embodiment peptide formed can be cleaved from the resin using chemicals selected from but not limited to difluoroacetic acid, trifluoro acetic acid and the like.

In one embodiment the purification process of GLP-1 analogs selected from D-Liraglutide or D-Semaglutide can be carried out by the processes well known in art. Purification process can be selected from but not limited to preparative reverse phase HPLC, ion exchange chromatography, size exclusion chromatography affinity chromatography and the like.

The synthetic analogs of GLP-1 namely D-Liraglutide and D-Semaglutide provided in accordance with the present disclosure can have better pharmacokinetic profile as compared to the native liraglutide and semaglutide respectively.

The GLP-1 analogs provided in accordance with the present disclosure that is D-Liraglutide and D-Semaglutide may be advantageous for relieving the patient's burden for example by reducing the frequency of administration of the dosage form comprising the analog of Liraglutide or Semaglutide.

The GLP-1 analog of the present disclosure that is D-Liraglutide or D-Semaglutide can be used in the treatment of metabolic disorders such as diabetes and obesity.

The GLP-1 analogs of the present disclosure that is D-Liraglutide or D-Semaglutide can offer advantage over the respective conventional GLP-1 in that it may be administered at a lower frequency, thus providing convenience to patients and thereby increasing patient compliance, further providing an effective blood glucose control over a longer period of time.

The GLP-1 analog in accordance with the present disclosure D-Liraglutide or D-Semaglutide can be a long acting analog suitable for once weekly or biweekly or monthly administration.

The GLP-1 analog D-Liraglutide or D-Semaglutide may be present in the form of base or in the form of its salts or mixtures thereof. Representative example of salts includes salts with suitable inorganic acids such as hydrochloric, hydrobromic, and the like. Representative examples of salts also include salts with organic acids such as formic acid, acetic acid, propionic acid, lactic acid, tartaric acid, ascorbic acid and the like. Representative examples of salts also include salt with base such as triethanolamine, diethylamine, meglumine, arginine, alanine, leucine, diethylethanolamine, triethylamine, tromethamine, choline, trimethylamine, taurine, benzamine, methylamine, dimethylamine, trimethylamine, methylethanolamine, propylamine, isopropylamine, adenine, guanine, cytosine, thymine, uracil, thymine, xanthine, hypoxanthine and like.

However, a person skilled in the art would appreciate that any other synthetic moiety, not capable of being degraded by DPP-IV, as known to those of ordinary skill in the art, can be used without departing from the scope and spirit of the instant disclosure.

In another embodiment, the GLP-1 analog provided in accordance with the present disclosure that is D-Liraglutide or D-Semaglutide may be respectively provided in the form of a respective lyophilized mixture comprising the D-Liraglutide or D-Semaglutide with parenterally acceptable amine base. The lyophilized mixture may be prepared by mixing the GLP-1 analog D-Liraglutide or D-Semaglutide or a pharmaceutically acceptable salt thereof and a parenterally acceptable amine base in water for injection to form a solution and lyophilizing the solution to form the lyophilized mixture. The parenterally acceptable amine base may be selected from triethanolamine, diethylamine, meglumine, ornithine, lysine, arginine, alanine, leucine, diethylethanolamine, olamine, triethylamine, tromethamine, glucosamine, choline, trimethylamine, taurine, benzamine, trimethyl ammonium hydroxide, epolamine methylamine, dimethylamine, trimethylamine, methylethanolamine, propylamine, isopropylamine, and like.

The present disclosure also provides use of GLP-1 analog provided in accordance with the present disclosure that is D-Liraglutide and D-Semaglutide of the present disclosure in the treatment of metabolic diseases. In a preferred embodiment, the Semaglutide analog of the present disclosure can be suitable for use in the treatment of diabetes. In another embodiment, the Semaglutide analog of the present disclosure may be suitable for use in the treatment of obesity.

In another embodiment, the GLP-1 analog of the present disclosure that is D-Liraglutide or D-Semaglutide can be suitable for use in reducing blood glucose levels in a patient in need thereof for a period of at least one week.

In another embodiment, the present disclosure provides a suitable dosage form for the administration of the GLP-1 analog of the present disclosure that is D-Liraglutide or D-Semaglutide by the oral or parenteral route.

The GLP-1 analog of the present disclosure that is D-Liraglutide or D-Semaglutide may be formulated into a suitable parenteral dosage form. The GLP-1 analog D-Liraglutide or D-Semaglutide of the present disclosure or the composition comprising the same, or the dosage form comprising the same may be administered by subcutaneous or intramuscular injection.

The GLP-1 analog of the present disclosure D-Liraglutide or D-Semaglutide may be formulated into a suitable oral dosage form. The GLP-1 analog of the present disclosure D-Liraglutide or D-Semaglutide, or the composition comprising the same, or the oral dosage form comprising the same may be administered by orally at a frequency depending upon the need of the subject requiring the administration of GLP-1.

The GLP-1 analog of the present disclosure D-Liraglutide or D-Semaglutide can provide a maintenance of therapeutic levels after single administration over an extended period of time which may be for a week or two weeks or for a month.

The GLP-1 analog or the of the present disclosure D-Liraglutide or D-Semaglutide can be used for the treatment of diabetes by administration of GLP-1 analog or the composition, or the dosage form comprising the same once weekly, once biweekly or once monthly.

In another embodiment, the present disclosure provides a method of reducing glucose levels in a patient in need thereof, comprising administering GLP-1 analog of the present disclosure D-Liraglutide or D-Semaglutide in therapeutically effective amount.

According to another embodiment the present invention provides pharmaceutical composition comprising GLP-1 analog of the present disclosure D-Liraglutide or D-Semaglutide as an active ingredient, together with one or more pharmaceutically acceptable carriers or excipients.

According to another embodiment composition can be prepared by mixing one or more analogs described herein, or pharmaceutically acceptable salts or tautomers thereof, with pharmaceutically acceptable carriers or the like, to treat or ameliorate a variety of GLP-1 related conditions. The pharmaceutical compositions of the present disclosure can be manufactured by methods well known in the art such as conventional granulating, mixing, dissolving, encapsulating, lyophilizing, emulsifying or levigating processes, among others. The compositions can be in the form of, for example, granules, powders, tablets, capsule syrup, suppositories, injections, emulsions, elixirs, suspensions or solutions. The instant compositions can be formulated for various routes of administration, for example, by oral administration, transmucosal administration, rectal administration, topical administration or subcutaneous administration as well as intrathecal, intravenous, intramuscular, intraperitoneal, intranasal, intraocular or intraventricular injection. The compound or compounds of the instant invention can also be administered in a local rather than a systemic fashion, such as injection as a sustained release formulation.

According to another embodiment analog of GLP-1 of the present disclosure D-Liraglutide or D-Semaglutide can be used alone or in combination with one or more additional therapeutically active agent.

In one embodiment, the invention provides methods of treating a GLP-1 mediated disease, disorder or syndrome in a subject comprising administering an effective amount of GLP-1 analog of the present disclosure D-Liraglutide or D-Semaglutide.

In another embodiment, the invention provides methods of treating a GLP-1 mediated disease, disorder or syndrome in a subject comprising administering an effective amount of GLP-1 analog D-Liraglutide or D-Semaglutide, wherein the disease is Type 2 diabetes, Type 1 diabetes, impaired glucose tolerance, hyperglycemia, metabolic syndrome (syndrome X and/or insulin resistance syndrome), glycosuria, metabolic acidosis, arthritis, cataracts, diabetic neuropathy, diabetic nephropathy, diabetic retinopathy, diabetic cardiomyopathy, obesity, conditions exacerbated by obesity, hypertension, hyperlipidemia, atherosclerosis, osteoporosis, osteopenia, frailty, bone loss, bone fracture, acute coronary syndrome, short stature due to growth hormone deficiency, infertility due to polycystic ovary syndrome, anxiety, depression, insomnia, chronic fatigue, epilepsy, eating disorders, chronic pain, alcohol addiction, diseases associated with intestinal motility, ulcers, irritable bowel syndrome, inflammatory bowel syndrome or short bowel syndrome.

In another embodiment, the invention provides use of GLP-1 analog D-Liraglutide or D-Semaglutide for the treatment of the diseases selected from Type 2 diabetes, Type 1 diabetes, impaired glucose tolerance, hyperglycemia, metabolic syndrome (syndrome X and/or insulin resistance syndrome), glycosuria, metabolic acidosis, arthritis, cataracts, diabetic neuropathy, diabetic nephropathy, diabetic retinopathy, diabetic cardiomyopathy, obesity, conditions exacerbated by obesity, hypertension, hyperlipidemia, atherosclerosis, osteoporosis, osteopenia, frailty, bone loss, bone fracture, acute coronary syndrome, short stature due to growth hormone deficiency, infertility due to polycystic ovary syndrome, anxiety, depression, insomnia, chronic fatigue, epilepsy, eating disorders, chronic pain, alcohol addiction, diseases associated with intestinal motility, ulcers, irritable bowel syndrome, inflammatory bowel syndrome or short bowel syndrome.

While the foregoing describes various embodiments of the disclosure, other and further embodiments of the disclosure may be devised without departing from the basic scope thereof. The scope of the disclosure is determined by the claims that follow. The disclosure is not limited to the described embodiments, versions or examples, which are included to enable a person having ordinary skill in the art to make and use the invention when combined with information and knowledge available to the person having ordinary skill in the art.

### EXAMPLES

The present invention is further explained in the form of following examples. However, it is to be understood that the following examples are merely illustrative and are not to be taken as limitations upon the scope of the invention.

### Example 1

### Synthesis of D-Liraglutide

### Step 1: Anchoring of Fmoc-Gly-CTCon Resin

Fmoc-Gly-CTC resin with a substitution degree of 0.35 mmol/g was weighed and added to the Solid-phase reaction column. Subsequently, the Fmoc-Gly-CTC resin was washed twice using DMF, and swollen in DMF for 30 min.

### Step 2: Deprotecting the Amino Acid

Fmoc protection was removed by 20% piperidine, and the resin was then washed for 4 times with DMF and twice by DCM. The resin was tested by ninhydrin test, in which the removal of Fmoc was indicated by the appearance of color of the resin.

### Step 3: Sequential Coupling of Other Fmoc-Protected Amino Acids

Fmoc-Arg(Pbf)-OH (6.0 mmol), HOBt (7.2 mmol), DIC (7.2 mmol) were dissolved in a mixed solution of DCM and DMF in a volume ratio of 1:1, loaded to the Solid-phase reaction column and reacted at room temperature for 2 h. The endpoint of the reaction was determined by ninhydrin test, in which the colorless and transparent resin indicated a complete reaction; while a color developed by the resin indicated an incomplete reaction, for which another 1 hr reaction was required. Such criteria were applied to the endpoint determination by ninhydrin test.
The above step 2 and corresponding amino acid coupling step were repeated, and based on the sequence of peptide backbone of D-Liraglutide, Fmoc-Arg(Pbf)OH, Fmoc-Gly-OH, Fmoc-Arg(Pbf)OH, Fmoc-Val-OH, Fmoc-Leu-OH, Fmoc-Trp(Boc)-OH, Fmoc-Ala-OH, Fmoc-Ile-OH, Fmoc-Phe-OH, Fmoc-Glu(OtBu)-OH, Fmoc-Lys(Dde)-OH, Fmoc-Ala-OH, Fmoc-Ala-OH, Fmoc-Gln(Trt)-OH, Fmoc-Glu(OtBu)-OH, Fmoc-Gly-OH, Fmoc-Leu-OH, Fmoc-Tyr(tBu)-OH, Fmoc-Ser(tBu)-OH, Fmoc-Ser(tBu)-OH, Fmoc-Val-OH, Fmoc-Asp(OtBu)-OH, Fmoc-Ser(tBu)-OH, Fmoc-Thr(tBu)-OH, Fmoc-Phe-OH, Fmoc-Thr(tBu)-OH, Fmoc-Phe-OH, Fmoc-Thr(tBu)-OH, Glu(OtBu)-OH, Fmoc-Gly-OH, Fmoc-D-Ala-OH and Boc-His(Trt)-OH were sequentially coupled.

### Step 4: Preparation of Dde Deprotected Resin Fragment

The resin fragment obtained after sequential coupling in above step-3 was added with a clear mixture of 3% Hydrazine hydrate in DMF lot-1 (10 vol). The suspension was gently agitated under nitrogen bubbling and mild stirring at 25-30° C. for 10 min. The solvent was drained and the resin was added with a clear mixture of 3% Hydrazine hydrate in DMF lot-2 (10 vol). The suspension was stirred at 25-30° C. for 10 min. The solvent was drained and the resin was washed with DMF (2×10 vol) and IPA (1×10 vol) and DMF (2×10 vol). Completion of the Dde-deprotection was confirmed by Kaiser colour test.

### Step 5: Coupling of Fmoc-Glu-OtBu and Palmitic Acid

### Step (5a): Coupling of Fmoc-Glu-OtBu

A clear mixture of Fmoc-Glu-OtBu (2.0 equiv), N,N-diisopropylcarbodiimide (DIC) (2.0 equiv) and 1-Hydroxybenzotriazole (HOBt) (2.0 equiv) in DMF (10 vol) was added to the resin. The suspension was gently agitated under nitrogen bubbling and mild stirring at 45-55° C. for 30 min. Progress of the reaction was monitored by Kaiser colour test. After completion of the reaction, the reaction solvent was drained and resin washed with DMF (4×10 vol).

### Step (5b): Fmoc-Deprotection

The resin was added with a clear mixture of 20% piperidine in DMF lot-1 (10 vol). The suspension was gently agitated under nitrogen bubbling and mild stirring at 25-30° C. for 10 min. The solvent was drained and the resin was added with a clear mixture of 20% piperidine in DMF lot-2 (10 vol). The suspension was stirred at 25-30° C. for 10 min. The solvent was drained and the resin was washed with DMF (2×10 vol) and IPA (1×10 vol) and DMF (2×10 vol). Completion of the Fmoc-deprotection was confirmed by Kaiser colour test.

### Step (5c): Coupling of Palmitic Acid

A clear mixture of Palmitic acid (2.0 equiv), N,N-diisopropylcarbodiimide (DIC) (2.0 equiv) and 1-Hydroxybenzotriazole (HOBt) (2.0 equiv) in DMF (10 vol) was added to the resin. The suspension was gently agitated under nitrogen bubbling and mild stirring at 45-55° C. for 30 min. Progress of the reaction was monitored by Kaiser colour test. After completion of the reaction, the reaction solvent was drained and resin washed with DMF (4×10 vol).

### Step 6: Preparation of D-Liraglutide

2-CTC resin bound protected Fragment obtained in step-5 was charged into a peptide synthesis flask. The resin was suspended in dichloromethane (DCM) (10 vol) without stirring for 10 min. The resin was added with a mixture of TFA:TIPS:DODT:Water (8.5:0.5:0.5:0.5 vol). The suspension was gently agitated under nitrogen bubbling and mild stirring at 25-30° C. for 3.0 h. The resin was filtered through a sintered funnel. The filtrate was added into a pre-cooled mixture of MTBE at 0-10° C. After complete addition, the reaction mixture was stirred for 1.0 h at 0-35° C. to precipitate an off-white solid. The precipitated solid was then filtered through a Buckner funnel and washed with MTBE. The suction dried solid was then dried in a vacuum oven at 35-40° C. till constant weight D-Liraglutide was obtained.

### Step 7: Purification of D-Liraglutide

### Step (7a): Purification-1

3.6 g D-Liraglutide obtained after global deprotection was dissolved in 300 mL buffer A, pH adjusted to 8.5-9.5 using ˜0.5 mL ammonium hydroxide soln. Below parameters were followed for purification:
Column specification: 250×50 mm SS
Media Specification: C-18 (3rd generation), 10μ, 100 A°
Mobile Phase A: 0.01M ammonium bicarbonate, Mobile Phase B: Acetonitrile,
Pooling criteria: Fractions having HPLC purity ≥85% and single maximum impurity ≤3% were pooled for purification 2.
Fractions having HPLC purity ≤85% and ≥60% were pooled for repurification.

### Step (7b): Purification-2

Pooled fractions from purification-1 having peptide content 900 mg was further diluted with equal amount of purified water and was purified as per following parameters:
Column specification: 250×50 mm SS
Media Specification: C-18 (3rd generation), 10μ, 100 A°
Mobile Phase A: 0.1% TFA in water, Mobile Phase B: Acetonitrile,
Pooling criteria: Fractions having HPLC purity ≥96% and single maximum impurity ≤0.5% were pooled for purification 3.
Fractions having HPLC purity ≤96% and ≥85% were pooled for repurification.

### Step (7c): Purification-3

Pooled fractions from purification-2 having peptide content 1200 mg was further diluted with equal amount of purified water and purified as follows:
Column specification: 250×50 mm SS
Media Specification: C-18 (3rd generation), 10μ, 100 A°
Mobile Phase A: 0.05% ammonium hydroxide in water, Mobile Phase B: Acetonitrile, Mobile Phase C: 3% ammonium acetate in water, Mobile Phase D: Purified water
Pooling criteria: Fractions having HPLC purity ≥98% and single maximum impurity ≤0.3% were pooled for concentration.
Fractions having HPLC purity ≤98% and ≥96% were pooled for repurification.
The pooled fractions from purification-3 were concentrated and subjected to lyophilization to obtain pure D-Liraglutide (I) as off-white to white powder.
D-Liraglutide obtained had HPLC Purity not less than 99.0% and isolated yield was in the range of 9-12%.

### Example 2

### Synthesis of D-Semaglutide

D-Semaglutide was synthesized as per the following process.

Step 1 to Step 4: The process described in Example 1 for synthesis of D-Liraglutide preparation as per steps 1 to 4 was followed.
Step 5: Coupling of Fmoc-PEG2-CH₂—COOH, Fmoc-PEG2-CH₂—COOH, Fmoc-Glu-OtBu and 18-tBu-18-Oxaoctadecanoic Acid

The coupling was carried out in a step wise manner as per the following scheme in stepwise manner:

### Step (5a): Coupling of Fmoc-PEG2-CH₂—COOH

A clear mixture of Fmoc-PEG2-CH₂—COOH (2.0 equiv), N,N-diisopropylcarbodiimide (DIC) (2.0 equiv) and 1-Hydroxybenzotriazole (HOBt) (2.0 equiv) in DMF (10 vol) was added to the resin. The suspension was gently agitated under nitrogen bubbling and mild stirring at 45-55° C. for 30 min. Progress of the reaction was monitored by Kaiser colour test. After completion of the reaction, the reaction solvent was drained and resin washed with DMF (4×10 vol).

### Step (5b): Fmoc-Deprotection

The resin was added with a clear mixture of 20% piperidine in DMF lot-1 (10 vol). The suspension was gently agitated under nitrogen bubbling and mild stirring at 25-30° C. for 10 min. The solvent was drained and the resin was added with a clear mixture of 20% piperidine in DMF lot-2 (10 vol). The suspension was stirred at 25-30° C. for 10 min. The solvent was drained and the resin was washed with DMF (2×10 vol) and IPA (1×10 vol) and DMF (2×10 vol). Completion of the Fmoc-deprotection was confirmed by Kaiser colour test.

### Step (5c): Coupling of Fmoc-PEG2-CH₂—COOH

A clear mixture of Fmoc-PEG2-CH₂—COOH (2.0 equiv), N,N-diisopropylcarbodiimide (DIC) (2.0 equiv) and 1-Hydroxybenzotriazole (HOBt) (2.0 equiv) in DMF (10 vol) was added to the resin. The suspension was gently agitated under nitrogen bubbling and mild stirring at 45-55° C. for 30 min. Progress of the reaction was monitored by Kaiser colour test. After completion of the reaction, the reaction solvent was drained and resin washed with DMF (4×10 vol).

### Step (5d): Fmoc-Deprotection

The resin was added with a clear mixture of 20% piperidine in DMF lot-1 (10 vol). The suspension was gently agitated under nitrogen bubbling and mild stirring at 25-30° C. for 10 min. The solvent was drained and the resin was added with a clear mixture of 20% piperidine in DMF lot-2 (10 vol). The suspension was stirred at 25-30° C. for 10 min. The solvent was drained and the resin was washed with DMF (2×10 vol) and IPA (1×10 vol) and DMF (2×10 vol). Completion of the Fmoc-deprotection was confirmed by Kaiser colour test.

### Step (5e): Coupling of Fmoc-Glu-OtBu

A clear mixture of Fmoc-Glu-OtBu (2.0 equiv), N,N-diisopropylcarbodiimide (DIC) (2.0 equiv) and 1-Hydroxybenzotriazole (HOBt) (2.0 equiv) in DMF (10 vol) was added to the resin. The suspension was gently agitated under nitrogen bubbling and mild stirring at 45-55° C. for 30 min. Progress of the reaction was monitored by Kaiser colour test. After completion of the reaction, the reaction solvent was drained and resin washed with DMF (4×10 vol).

### Step (5f): Fmoc-Deprotection

The resin was added with a clear mixture of 20% piperidine in DMF lot-1 (10 vol). The suspension was gently agitated under nitrogen bubbling and mild stirring at 25-30° C. for 10 min. The solvent was drained and the resin was added with a clear mixture of 20% piperidine in DMF lot-2 (10 vol). The suspension was stirred at 25-30° C. for 10 min. The solvent was drained and the resin was washed with DMF (2×10 vol) and IPA (1×10 vol) and DMF (2×10 vol). Completion of the Fmoc-deprotection was confirmed by Kaiser colour test.

Step (5g): Coupling of 18-tBu-18-Oxaoctadecanoic Acid

A clear mixture of 18-tBu-18-Oxaoctadecanoic acid (2.0 equiv), N,N-diisopropylcarbodiimide (DIC) (2.0 equiv) and 1-Hydroxybenzotriazole (HOBt) (2.0 equiv) in DMF (10 vol) was added to the resin. The suspension was gently agitated under nitrogen bubbling and mild stirring at 45-55° C. for 30 min. Progress of the reaction was monitored by Kaiser colour test. After completion of the reaction, the reaction solvent was drained and resin washed with DMF (4×10 vol).

### Step 6: Preparation of D-SEMAGLUTIDE

2-CTC resin bound protected Fragment obtained in step 5 was charged into a peptide synthesis flask. The resin was suspended in dichloromethane (DCM) (10 vol) without stirring for 10 min. The resin was added with a mixture of TFA:TIPS:DODT:Water (8.5:0.5:0.5:0.5 vol). The suspension was gently agitated under nitrogen bubbling and mild stirring at 25-30° C. for 3.0 h. The resin was filtered through a sintered funnel. The filtrate was added into a pre-cooled mixture of MTBE at 0-10° C. After complete addition, the reaction mixture was stirred for 1.0 h at 0-35° C. to precipitate an off-white solid. The precipitated solid was then filtered through a Buckner funnel and washed with MTBE. The suction dried solid was then dried in a vacuum oven at 35-40° C. till constant weight to provide D-Semaglutide.

### Example 3

### Purification of (L-Ala)-Liraglutide and (D-Ala)-Liraglutide

A method for purifying crude both (L-Ala)-Liraglutide (native) and (D-Ala)-Liraglutide obtained from solid-phase synthesis, which is characterized by comprising the following steps:

Step 1: A solution of crude liraglutide is obtained by dissolving 100 mg crude liraglutide obtained from solid-phase synthesis in 0.01M Ammonium bicarbonate with 25% Ammonia solution and filtered with 0.2-micron filter.
Step 2: The solution of crude liraglutide both (L-Ala)-Liraglutide and (D-Ala)-Liraglutide is subjected to a first HPLC purification using 10*250 mm Phenominex C18 (3gen.) 100 A, 10-micron column, and using 0.01M Ammonium bicarbonate as mobile phase A and acetonitrile as mobile phase B eluting at a gradient as mentioned in Table 1, and target peak is collected and analyzed with RP-HPLC for purity and content.

TABLE 1
HPLC depicting elution of mobile phase B for crude Liraglutide

| Time | % B |
|---|---|
| 0 | 10 |
| 15 | 10 |
| 45 | 30 |
| 65 | 30 |
| 80 | 35 |

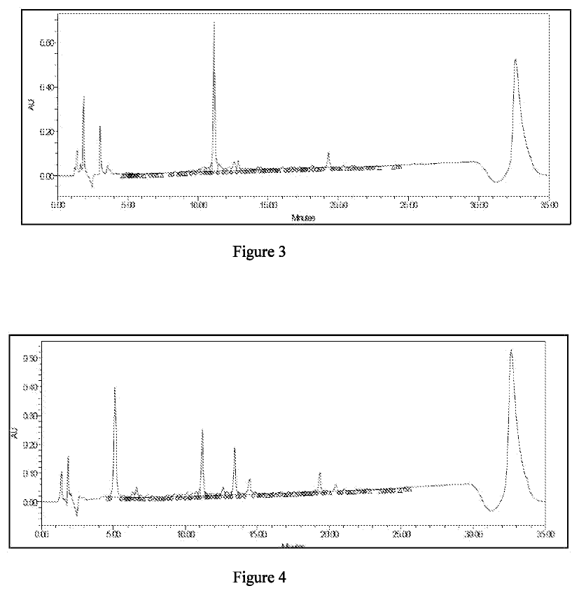

The RP-HPLC profile for crude Liraglutide and D-Liraglutide with purity of 50.4% and 15.1% respectively are shown in FIGS. 3 and 4.

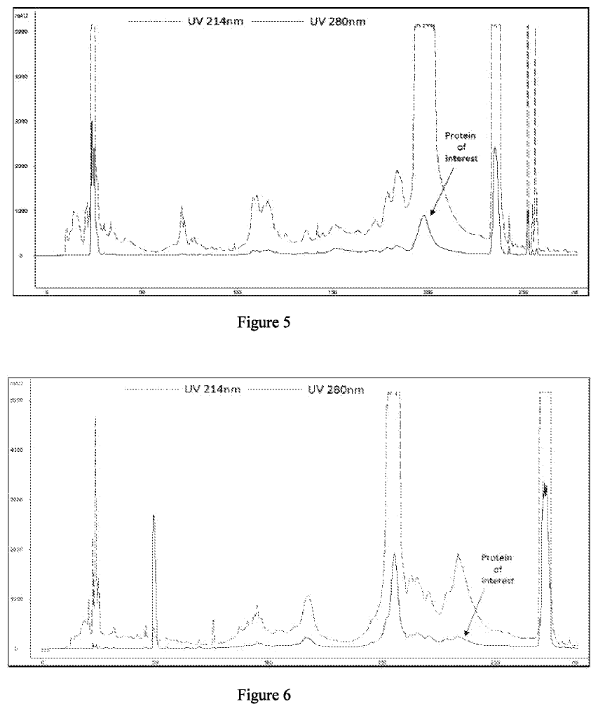

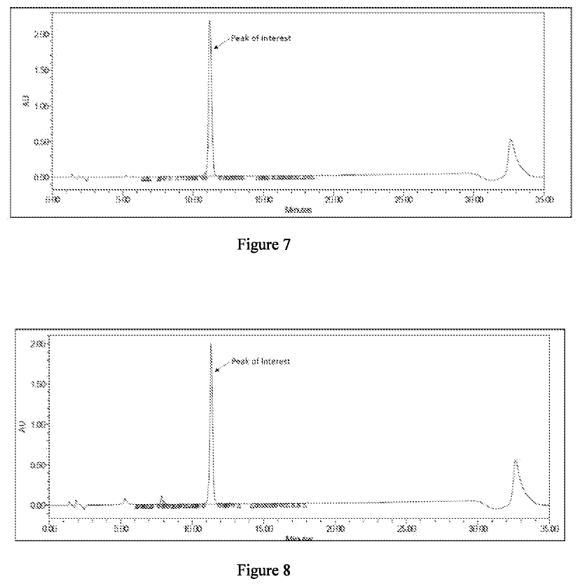

The chromatogram profile with peak of interest for both Liraglutide and D-Liraglutide are described in FIG. 5 and FIG. 6. The freeze dried purified fractions pooled for Liraglutide and D-Liraglutide shows RP-HPLC purity of 93.1% and 90.0% respectively (FIG. 7 and FIG. 8 respectively). Details are depicted in Table 2 below:

TABLE 2
Summary of purification process yield

| | | | |
|---|---|---|---|
| | | Yield/Recovery | |
| Sample details | Total protein | (%) | Purity |

Purification of Liraglutide

| | | | | |
|---|---|---|---|---|
| (L-Ala)- Liraglutide | 12 | mg | 12.0 | 50% |
| crude | | | | |
| Chrom 1 purified | 4 | mg | 33.3 | 93.1% |
| sample | | | | |

Purification of D-Liraglutide

| | | | | |
|---|---|---|---|---|
| (D-Ala)- Liraglutide | 5 | mg | 5.0 | 15% |
| crude | | | | |
| Chrom 1 purified | 0.5 | mg | 10 | 88.2% |
| sample | | | | |

### Example 4

### Biological Characterization of Liraglutide and D-Liraglutide

The in-vitro potency of in-house product is determined based on the stimulation of adenylate cyclase activity in the rat thyroid c-cell line 6-23 (Clone 6) (ATCC® CRL-1607™). Activation of the GLP-1 receptor initiates a cascade event which culminates in an intracellular rise in cAMP concentration was determined and compared with the RMP (Victoza) using cAMP ELISA kit.

The statistical analysis was done using Graph Pad Prism software.

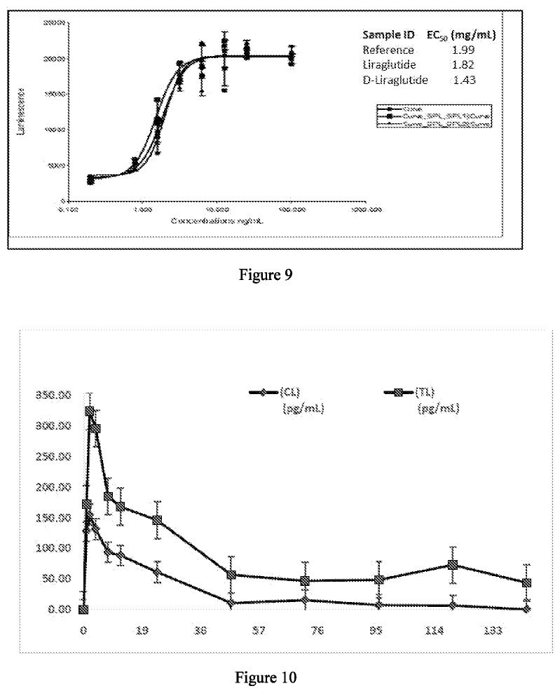

The EC50 value for Reference (Victoza) observed was 1.99 ng/mL and that of synthetic Liraglutide and D-Liraglutide were 1.82 ng/ml and 1.43 ng/mL, respectively as described in FIG. 9.

### Example 5

### Pharmacokinetic (PK) Analysis of Liraglutide and D-Liraglutide in Diabetes Mellitus (DM-2) Wistar Rats

Streptozotocin (STZ), preferentially toxic to pancreatic beta cells, is commonly used to model Type-2 diabetes mellitus (DM) in numerous species, including Wistar rat. 12-14 week-old male Wistar rats (300-380 g body weight) were selected for the study. Rats were randomly distributed to different groups based on ad-lib fed blood glucose and body weight. The diabetes mellitus (DM) was induced in Wister rats by single intraperitoneal injection of streptozotocin at 60 mg/kg STZ (n=6) in both control and test group for two weeks.

Basal glucose levels were recorded for all the animals before injecting STZ. All animals were again evaluated for the elevated blood levels after 15 days of STZ treatment. After the confirmation of high glucose levels animals were treated with single dose (5 mg/kg) of Liraglutide and D-Liraglutide by subcutaneous route.

Blood samples were collected at 0 (Pre-dose), 1, 2, 4, 8, 12, 24, 48, 72, 96, 120 and 144 h, post dose. At each time point, approximately, 0.3 mL of blood was withdrawn through retro orbital plexus under light isoflurane anesthesia in a labeled microfuge tube. All blood samples were centrifuged at 7000 rpm for 5 min at set temperature of 4° C. After centrifugation serum was separated and stored at −80° C. for further analysis.

The ELISA was performed for the quantitative measurement of the Liraglutide in the serum samples using Cloud-Clone Corp. (CEV769Ge 96 Tests) kit. The kit works on competitive inhibition enzyme inhibition assay technique. All the serum samples of both groups, Liraglutide and D-Liraglutide were diluted 1:100 using sample dilution buffer and tested in ELISA for the presence of Liraglutide at various time points using standard graph.

The data obtained after the complete ELISA and statistical analysis was run through the PK solver software using Non compartmental model for the PK parameters (T_{1/2}, Cₘₐₓ, Tₘₐₓ, AUC₀₋ₜ and MRT) determination as shown in Table 3 and FIG. 10.

TABLE 3
PK parameters comparison table for both
groups (Liraglutide and D-Liraglutide)

| PK Parameters | Liraglutide | D-Liraglutide |
|---|---|---|
| t½ (h) | 24.77 | 54.55 |
| Tmax (h) | 2 | 2 |
| Cmax (pg/ml) | 155.08 | 324.81 |
| AUC 0-inf (pg/ml*h) | 4055.04 | 15673.63 |
| MRT (h) | 34.83 | 89.71 |

The D-Liraglutide was studied in order to investigate if the delayed absorption allowed an extended dosing interval without affecting the predicted clinical efficacy, thus reducing the cost of treatment, enhancing prescription compliance, and favouring animal welfare. The results show that significant differences were detected between Liraglutide and D-Liraglutide for (half-life (T½) 24.77 hr to 54.44 hr for the SC administration, resp.). Absolute bioavailability was significantly high in case of D-Liraglutide as the AUC values were found to be 3 times higher than the Liraglutide. The higher Cmax value for D-Liraglutide also supports the above finding.

### Example 6

### Oral Bioavailability of D-Liraglutide as Against the Subcutaneous Route

The second phase of pharmacokinetic study was conducted to understand the oral bioavailability of D-Liraglutide as against the subcutaneous route. Usually, proteins and peptides show poor oral bioavailability owing to extensive degradation by enzymes in the gastrointestinal tract, as well as limited permeability across the gastrointestinal mucosa. Their oral bioavailability to less than 1%.

Healthy male adult rats of age (7-9 weeks) were randomized in to two groups. Control groups was administered 6 mg/kg of Victoza subcutaneously and test group was administered orally with 15 mg/kg of test molecule that is D-Liraglutide. Blood samples were collected as per Table 4 to estimate the content of liraglutide in blood for PK comparison.

TABLE 4
Liraglutide PK studies

| | |
|---|---|
| Animal | Male SD rats (~300 gm weight) |
| Number of groups | 2 |
| Number of | 6 |
| animals/group | |
| Route of | Subcutaneous for G1 and P.O. (Oral capsules) for |
| administration | G2 |
| Dose | Single dose of 6 mg/kg for SC and 15 mg/kg for Oral |
| Dose volume | 1 mL/KG (SC) and 1 capsule/animal in G4 |
| Time points | 0, 1, 2, 4, 8, 12, 24, 48, 72, 120, 144 & 168 Hours |
| Withdrawal | 300 μL blood from retro orbital plexus |
| Sample prep | Plasma samples to be prepared and immediately |
| | frozen in Liquid N2/−80° C. freezer |
| Shipment | All time points plasma samples were analysed till 24 |
| | hours |

### Liraglutide Estimation in Plasma Samples:

50× doses of Liraglutide in SD Rat Plasma was prepared and Diluted it in Assay Buffer (HBSS with IMBX, MgCl₂ and Ro). Then cell overexpressing GLP-1R were [(CHOK1/GLP1/Gα15) cat no M00451 Lot no: R10081093-12)] were harvested by trypsinization and centrifuged. These cells were then washed with Assay Buffer. Cells at 15 k/well/15 uL were seeded and 15 μL of 2× doses were added and plate was incubated for 30 minutes at 37° C. for 30 min. cAMP production was then estimated using cAMP estimation kit (Promega cAMP-Glo™ Max Assay Cat no: V1682). Post 30 minutes' incubation Protein Kinase A 20 μL solution was added to the plate and incubated for 20 minutes at RT. Then 50 μL of substrate was added to plate for 10 Minutes at RT

Luminescence was read on the plate reader. Liraglutide content in the plasma was back calculated using calibration curve generated on each run as per Table 5. For back calculation Gen 5 software was used. Preclinical PK Parameters of Liraglutide are presented in Table 6.

TABLE 5
Table of Calibration curve

| Liraglutide (pg/mL) | Back calculated actual | |
|---|---|---|
| Calibration Curve | concentration Avg | |
| concentration | (pg/mL) | % Recovery |
| 200 | 192 | 96 |
| 100 | 101 | 101 |
| 50 | 52 | 103 |
| 25 | 25 | 98 |
| 12.5 | 13 | 106 |
| 6.25 | 5 | 84 |
| 3.125 | Below limit | NA |

TABLE 6
Preclinical PK Parameters of Liraglutide
PK Parameters

| | Animal | Cₘₐₓ | AUC | Tₘₐₓ |
|---|---|---|---|---|
| Group | Number | (ng/mL) | (ng/mL*h) | (hour) |
| D Lira | R1 | 1.714 | 9.536 | 0.24 |
| | R3 | 7.183 | 151.333 | 2.49 |
| | R5 | 1.740 | 11.593 | 2.26 |
| | R6 | 1.341 | 9.484 | 1.10 |
| | Avg | 2.995 | 45.486 | 1.5 |
| Victoza | R1 | 65.8 | 1317.0 | 7.4 |
| | R3 | 635.7 | 13626.4 | 7.7 |
| | R4 | 2336.5 | 29033.4 | 3.3 |
| | R5 | 171.5 | 3675.8 | 7.9 |
| | R6 | 3808.5 | 41915.1 | 3.0 |

Peptide drugs always pose challenge in oral delivery due to digestive enzymatic degradation, hydrophobicity etc. Physiologically active form of GLP-1 is required for 5-10 minutes, also, concentration of GLP-1 required for glucose excursion is in single or lower double digit pM. Even after use of DPP-IV inhibitor circulating GLP-1 concentration reaches up to 50-60 pM, which is proved to clinically significant.

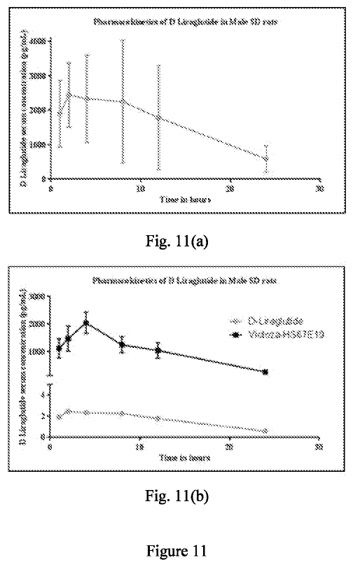

PK Profile of Orally Administered D-Liraglutide and Subcutaneously administered Victoza is shown in FIG. 11. Orally administered D-liraglutide showed cmax of 1.5 ng/ml correspond to 400 pM. Since physiologically GLP1 R activation is required to post prandial, 90-150 minutes. And circulating levels 60 pM of GLP-1 are sufficient to improve glucose tolerance. D-Liraglutide 400 pM levels which we observed in 3 animals may be therapeutically viable option. Even after consideration of lower potency (2-5 fold) of liraglutide compared to D-Liraglutide circulating concentration in this study may be sufficient to produce significant improvement in glucose tolerance.

1. (canceled)

2. An analog of liraglutide wherein an amino acid L-Alanine at position 2 of the native liraglutide amino acid sequence is replaced with D-Alanine, wherein the analog is D-Liraglutide.

3. An analog of semaglutide, wherein an amino acid Aib (Amino isobutyric acid) at position 2 of the native is replaced with D-Alanine, wherein the analog is D-Semaglutide.

4. A process for the preparation of Analog selected from D-Liraglutide and D-Semaglutide, in which the process comprises steps of:
a) anchoring Fmoc-Gly-OH to a resin and capping it;
b) selectively deprotecting the amino group;
c) sequential coupling of the fragments Fmoc-Arg(Pbf)OH, Fmoc-Gly-OH, Fmoc-Arg(Pbf)OH, Fmoc-Val-OH, Fmoc-Leu-OH, Fmoc-Trp(Boc)-OH, Fmoc-Ala-OH, Fmoc-Ile-OH, Fmoc-Phe-OH, Fmoc-Glu(OtBu)-OH, Fmoc-Lys(Dde)-OH, Fmoc-Ala-OH, Fmoc-Ala-OH, Fmoc-Gln(Trt)-OH, Fmoc-Glu(OtBu)-OH, Fmoc-Gly-OH, Fmoc-Leu-OH, Fmoc-Tyr(tBu)-OH, Fmoc-Ser(tBu)-OH, Fmoc-Ser(tBu)-OH, Fmoc-Val-OH, Fmoc-Asp(OtBu)-OH, Fmoc-Ser(tBu)-OH, Fmoc-Thr(tBu)-OH, Fmoc-Phe-OH, Fmoc-Thr(tBu)-OH, Fmoc-Phe-OH, Fmoc-Thr(tBu)-OH, Glu(OtBu)-OH, Fmoc-Gly-OH, Fmoc-D-Ala-OH and Boc-His(Trt)-OH;
d) removing of the lysine side chain protecting group Dde, followed by coupling with i) Fmoc-Glu-OtBu for D-Liraglutide, followed by Fmoc deprotection and coupling with palmitic acid; or with ii) Fmoc-PEG2-CH₂—COOH for D-Semaglutide followed by Fmoc deprotection and coupling with oxaoctadecanoic acid and
e) cleaving the peptide from the resin to obtain linear D-Liraglutide or D-Semaglutide.

5. (canceled)

6. The process as claimed in claim 3, wherein the process optionally comprises purification of D-Liraglutide or D-Semaglutide to provide purified D-Liraglutide or D-Semaglutide respectively.

7. The process as claimed in claim 3, wherein the coupling agent is selected from 1-Hydroxybenzotriazole (HOBt), N,N-diisopropylcarbodiimide (DIC), Hexafluorophosphate Benzotriazole Tetramethyl Uronium (HBTU), N,N-Diisopropylethylamine (DIPEA), benzotriazol-1-yl-oxy-tris(dimethyl-amino)-phosphonium hexafluorophosphate (BOP), and O-(7-azabenzotriazol-1-yl)-1,1,3,3-tetramethyluronium hexafluorophosphate (HATU).

8. The process as claimed in claim 3, wherein the solvent for coupling reaction is selected from Dimethylformamide (DMF), pyridine, acetic anhydride, methanol, ethanol, isopropanol, dichloroethane, 1,4-dioxane, 2-methyl tetrahydrofuran, N-methyl-2-pyrrolidinone (NMP), ethyl acetate, acetonitrile, and acetone.

9. A pharmaceutical composition comprising GLP-1 analog as claimed in claim 1 or 2, as an active ingredient, together with one or more pharmaceutically acceptable carriers or excipients.

10. The composition claimed in claim 7, wherein the route of administration is oral or parenteral.

11. A method of reducing glucose levels in a patient in need thereof, comprising administering GLP-1 analog as claimed in claim 1, or 2 in therapeutically effective amount.

12. A method of treatment of GLP-1 mediated disease, disorder or syndrome in a subject comprising administering an effective amount of GLP-1 analog as claimed in claim 1, or 2

13. The method as claimed in claim 10, wherein the disease is selected from Type 2 diabetes, Type 1 diabetes, impaired glucose tolerance, hyperglycemia, metabolic syndrome (syndrome X and/or insulin resistance syndrome), glycosuria, metabolic acidosis, arthritis, cataracts, diabetic neuropathy, diabetic nephropathy, diabetic retinopathy, diabetic cardiomyopathy, obesity, conditions exacerbated by obesity, hypertension, by perlipidemia, atherosclerosis, osteoporosis, osteopenia, frailty, bone loss, bone fracture, acute coronary syndrome, short stature due to growth hormone deficiency, infertility due to polycystic ovary syndrome, anxiety, depression, insomnia, chronic fatigue, epilepsy, eating disorders, chronic pain, alcohol addiction, diseases associated with intestinal motility, ulcers, irritable bowel syndrome, inflammatory bowel syndrome or short bowel syndrome.

14. The method as claimed in claim 11, wherein the disease is selected from Diabetes and Obesity.

15. Use of GLP-1 analog as claimed in claim 1, or 2, for the treatment of the disease selected from Type 2 diabetes, Type 1 diabetes, impaired glucose tolerance, hyperglycemia, metabolic syndrome (syndrome X and/or insulin resistance syndrome), glycosuria, metabolic acidosis, arthritis, cataracts, diabetic neuropathy, diabetic nephropathy, diabetic retinopathy, diabetic cardiomyopathy, obesity, conditions exacerbated by obesity, hypertension, hyperlipidemia, atherosclerosis, osteoporosis, osteopenia, frailty, bone loss, bone fracture, acute coronary syndrome, short stature due to growth hormone deficiency, infertility due to polycystic ovary syndrome, anxiety, depression, insomnia, chronic fatigue, epilepsy, eating disorders, chronic pain, alcohol addiction, diseases associated with intestinal motility, ulcers, irritable bowel syndrome, inflammatory bowel syndrome or short bowel syndrome.
